(19) United States
(12) Patent Application Publication
(10) Pub. No.: US 2013/0133289 A1
(43) Pub. Date: May 30, 2013

(54) PROCESS FOR THE PREPARATION OF AMORPHUS VALGANCYCLOVIR HYDROCHLORIDE

(71) Applicants: Madhuresh Kumar Sethi, Hyderabad (IN); Vijendra Singh Ravat, Hyderabad (IN); Raja Krishna Yerramalla, Hyderabad (IN); Lakshminarayana Vemula, Hyderabad (IN); Jagan Mohana Rao Bontalakoti, Hyderabad (IN); Kartikeyan Ayyaran, Hyderabad (IN); Debashish Datta, Hyderabad (IN)

(72) Inventors: Madhuresh Kumar Sethi, Hyderabad (IN); Vijendra Singh Ravat, Hyderabad (IN); Raja Krishna Yerramalla, Hyderabad (IN); Lakshminarayana Vemula, Hyderabad (IN); Jagan Mohana Rao Bontalakoti, Hyderabad (IN); Kartikeyan Ayyaran, Hyderabad (IN); Debashish Datta, Hyderabad (IN)

(21) Appl. No.: 13/479,700

(22) Filed: May 24, 2012

Related U.S. Application Data
(63) Continuation of application No. PCT/IN2010/000751, filed on Nov. 18, 2010.

(30) Foreign Application Priority Data
Nov. 24, 2009 (IN) 2002/CHE/2009

(51) Int. Cl. C07D 473/18; B65B 31/02

(52) U.S. Cl. CPC C07D 473/18; B65B 31/024
USPC 53/434; 544/276; 428/402

(57) ABSTRACT

Amorphous valgancyclovir hydrochloride has a median particle size of below 100 μm. A process for the preparation of the compound includes dissolving valgancyclovir hydrochloride in at least one solvent, removing the solvents under moisture controlled conditions, and drying the wet mass.

This application is a continuation of PCT/IN2010/000751 filed Nov. 18, 2010 and claims priority to Indian patent application No. 2002/CHE/2009 filed on Nov. 24, 2009, the contents of which are incorporated by reference in their entirety.

### FIELD OF THE INVENTION

The present invention relates to a process for the preparation of an amorphous valgancyclovir hydrochloride.

The present invention also relates to an amorphous valgancyclovir hydrochloride having a median particle size below 100 μm.

### BACKGROUND OF THE INVENTION

Valcyte (Valganciclovir HCl tablets) contains valgancyclovir hydrochloride, a hydrochloride salt of the L-valyl ester of ganciclovir. Ganciclovir is a synthetic guanine derivative active against cytomegalovirus (CMV). Valganciclovir hydrochloride is a white to off-white crystalline powder with a molecular formula of C₁₄H₂₂N₆O₅.HCl. The chemical name of valgancyclovir hydrochloride is L-valine, 2-(2-amino-1,6-dihydro-6-oxo-purin-9-yl)-methoxy-3-ydroxy-1-propanyl ester monohydrochloride, the structure as shown in formula (I).

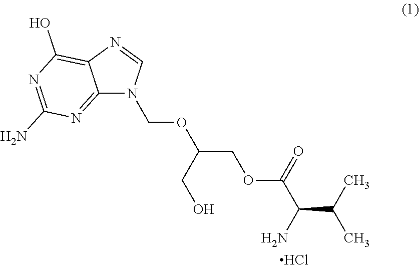 [SMILES: CC(C)[C@@H](N)C(=O)OCC(CO)OCn1cnc2c(O)nc(N)nc21 ; Cl]

European patent No. 375329 discloses ester prodrugs of ganciclovir i.e. valgancyclovir and its physiologically acceptable salts thereof having advantageous bioavailability when administered by an oral route. The patent also teaches about the process for the preparation of valgancyclovir and its physiologically acceptable salts thereof.

Published US application 20070129385 discloses an amorphous valgancyclovir hydrochloride and a process for the preparation of an amorphous valgancyclovir hydrochloride by using spray drying and azeotropic distillation techniques. This application also discloses the conversion of crystalline or mixture of crystalline and an amorphous valgancyclovir hydrochloride into an amorphous valgancyclovir hydrochloride.

Published US application 20090062538 discloses pure amorphous valgancyclovir hydrochloride and a process for the preparation of amorphous valgancyclovir hydrochloride by providing a solution, suspension or dispersion of valgancyclovir hydrochloride, either alone or in combination with one or more pharmaceutically acceptable carriers in a solvent, and removing the solvent from the solution using spray drying to provide pure amorphous valgancyclovir hydrochloride.

It was observed that the techniques employed, such as solvent removing techniques, in the prior art processes and other techniques always left some residual solvent, thus decreasing the purity of the final compound. To remove the solvent completely, the material has to be dried for a prolonged time which may again lead to the formation of impurities. The residual solvents present in the final product should be within the limits of ICH guidelines. Therefore, there is a need to develop an improved process for producing amorphous valganciclovir hydrochloride with desired particle size and suitable packing.

### OBJECT OF THE INVENTION

The main object of the present invention relates to an amorphous valgancyclovir hydrochloride having a median particle size below 100 μm under moisture controlled conditions.

### SUMMARY OF THE INVENTION

The main aspect of the present invention is to provide an amorphous valgancyclovir hydrochloride having a median particle size below 100 μm.

In another aspect, the present invention is to provide a process for the preparation of amorphous valgancyclovir hydrochloride having a median particle size below 100 pm, comprising the steps of:

a) dissolving valgancyclovir hydrochloride in a solvent or mixture of solvents,

b) removing the solvent(s) under moisture controlled conditions, and

c) drying the wet mass to isolate amorphous valgancyclovir hydrochloride having a median particle size below 100 μm under moisture controlled conditions.

In yet another aspect, the present invention is to provide a process for the preparation of amorphous valganciclovir hydrochloride having a median particle size below 100 μm comprising the steps of:

a) dissolving valgancyclovir hydrochloride in a solvent or mixture of solvents,

b) removing the solvent under moisture controlled conditions,

c) drying the wet mass to isolate amorphous valganciclovir hydrochloride having a median particle size below 250 μm under moisture controlled conditions, and

d) reducing the particle size to a median particle size below 100 μm.

In yet another aspect, the present invention is to provide an improved process for the preparation of amorphous valgancyclovir hydrochloride wherein the solvent is removed by using distillation, evaporation, spray drying or by agitated thin film evaporator under moisture controlled conditions.

In yet another aspect, the particles are optionally reduced by micronization to obtain the desired particle size under moisture controlled conditions.

In yet another aspect of the present invention is suitable packing to maintain polymorphic and chemical stability.

The amorphous valgancyclovir hydrochloride prepared, isolated and stored according to the present invention with desired particle size under moisture controlled conditions provides a stable amorphous valgancyclovir hydrochloride.

### BRIEF DESCRIPTION OF THE DRAWINGS

FIG. 1 is an X-ray powder diffraction (XRD) pattern of amorphous valganciclovir hydrochloride.

### DETAILED DESCRIPTION OF THE INVENTION

The present invention relates to an amorphous valgancyclovir hydrochloride having a median particle size below 100 μm.

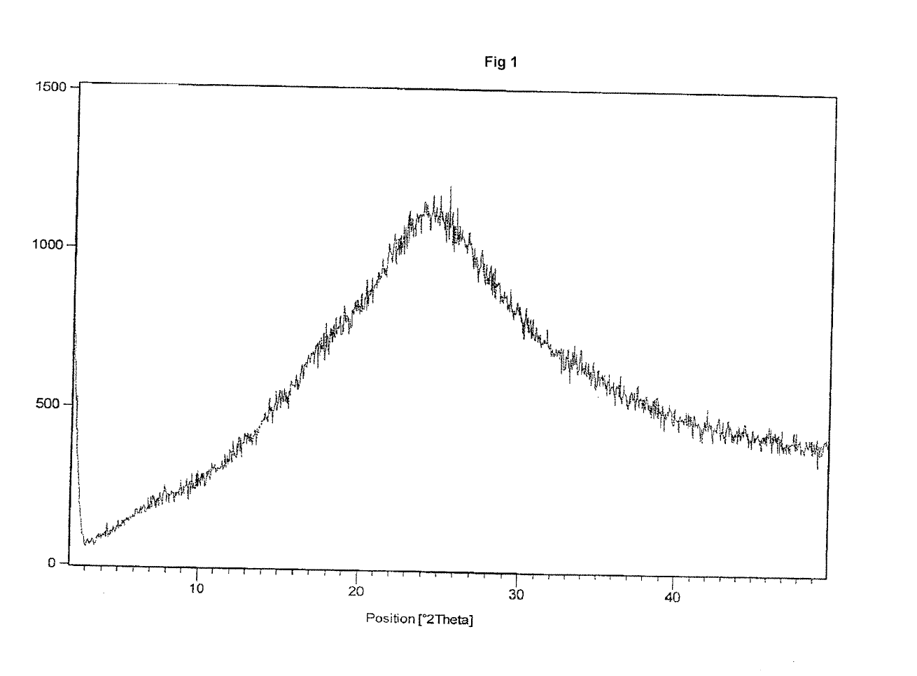

In one embodiment, an amorphous valgancyclovir hydrochloride disclosed in the present invention is confirmed by PXRD pattern shown in FIG. 1.

In another embodiment, the present invention relates to a process for the preparation of an amorphous valgancyclovir hydrochloride under moisture controlled conditions having a median particle size below 100 μm comprising the steps of:

a) dissolving valgancyclovir hydrochloride in a solvent or mixture of solvents,

b) removing the solvent, and

c) drying the wet mass to isolate an amorphous valgancyclovir hydrochloride having a median particle size below 100 μm.

According to the present invention, dissolving valganciclovir hydrochloride in a solvent or mixture of solvents. removing the solvent and drying the wet mass under vacuum yields amorphous valgancyclovir hydrochloride having a median particle size below 100 μm.

In yet another embodiment, the present invention is to provide a process for the preparation of an amorphous valgancyclovir hydrochloride under moisture controlled conditions having a median particle size below 100 μm comprising the steps of:

a) dissolving valgancyclovir hydrochloride in a solvent or mixture of solvents,

b) removing the solvent,

c) drying the wet mass to isolate an amorphous valgancyclovir hydrochloride having a median particle size below 250 μm, and

d) reducing the particle size to a median particle size below 100 μm.

According to the present invention, dissolving valgancyclovir hydrochloride in a solvent or mixture of solvents, removing the solvent and drying the wet mass under vacuum yields amorphous valgancyclovir hydrochloride having a median particle size below 250 μm which is micronized using conventional techniques to obtain amorphous valgancyclovir hydrochloride having a median particle size below 100 μm.

According to present invention, the suitable solvent for dissolution is selected from C1-C6 alcohols such as methanol, ethanol, n-propanol, isopropanol, n-butanol, isobutanol, and t-butanol; ketones such as acetone, propanone, and 2-butanone; esters such as ethyl acetate, n-propyl acetate, isopropyl acetate and n-butyl acetate; hydrocarbons, water and mixtures thereof. According to the present invention the solution is optionally filtered using conventional filter techniques, and preferably a micron filter.

According to the present invention, the solvent is removed under moisture controlled conditions using techniques such as distillation, evaporation, oven drying, tray drying, rotational drying, spray drying, freeze-drying, vacuum drying, thin film evaporation and the like.

In one embodiment, the solvent is removed using agitated thin film evaporator at a feed rate of preferably 1 to 50 ml/min at a temperature of about 10 to 100° C. using 0 to 200 mbar vacuum thus isolating wet valgancyclovir hydrochloride.

According to the present invention, the wet valgancyclovir hydrochloride is dried under vacuum. The vacuum used for drying is preferably below 100 m bar at a temperature of about 30-100° C. to obtain amorphous valgancyclovir hydrochloride with desired median particle size of below 100 μm.

According to the present invention the valgancyclovir hydrochloride particle size is optionally reduced using conventional techniques such as reduction by ball milling, roller milling, micronizing, hammer milling, jet milling, grinding and the like to get the desired particle size. In one embodiment, the particles are optionally micronized to obtain the median particle size of below 100 μm.

In another embodiment, the process according to the present invention yields an amorphous valgancyclovir hydrochloride having a diastereomeric ratio in the range of about 45:55 to 55:45.

In another embodiment, amorphous valgancyclovir hydrochloride is dried at critical condition to maintain loss on drying (LOD) content in the range of about 1-3%.

In another embodiment, amorphous valgancyclovir hydrochloride prepared according to the present invention has residual solvent content of less than about 3000 ppm. According to the present invention the residual solvent is methanol.

In another embodiment, the process according to the present invention yields an amorphous valgancyclovir hydrochloride having enantiomeric purity more than about 98.5%.

In another embodiment, amorphous valgancyclovir hydrochloride material is packed in a HMLDPE bag under vacuumised nitrogen sealing at 30±5% RH. Then it is inserted in a triple laminated aluminum bag under vacuumised nitrogen sealing with two silica gel sachets and one molecular sieve sachet. Again it is inserted in triple laminated aluminum bag under vacuumised nitrogen sealing with two silica gel sachets and one molecular sieve sachet. Such bags are further packed in high density polyethylene (“HDPE”) containers.

In accordance with the present invention, the particle size distributions were measured by laser diffraction utilizing a Malvern Mastersizer Scirocco 2000 (A). The XRD-patterns were obtained on a PANalytical X′Pert PRO Powder X-ray diffractometer with X′pert Data Collector software employing the following parameters:

| Instrument used | Powder X-ray Diffractometer |
|---|---|
| Make and Model | PANalytical X'PERT PRO MPD |
| Radiation | Cu Ka1 (I = 1.540598 A) |
| Detector | X'Celerator |
| Voltage (kV), Current (mA) | 40, 30 |
| K beta Filter | Nickel |
| Scan mode | Continuous |
| Angular range (2q) | 2-50 |
| Scan step (2q) | 0.033 |
| Step time (seconds) | 50 |

In order to provide a fuller understanding of the invention, the following examples are set forth. These examples are for the purpose of illustration only and are not to be construed as limiting the scope of the invention in any way.

### EXAMPLES

### Examples 1

### Preparation of Amorphous Valgancyclovir Hydrochloride

50 g Valgancyclovir hydrochloride was dissolved in methanol (850 ml) and the reaction mixture stirred to get a clear solution. The solution was passed through a micron filter to get a particle clear solution. The clear solution containing valganciclovir hydrochloride was evaporated using an agitated thin film evaporator at a feed rate of about 2 to 10 ml/min at 60° C. at about 100 mbar vacuum, thus isolating a wet powdered form of amorphous valganciclovir hydrochloride under moisture controlled conditions. The wet mass was dried in a vacuum oven to afford amorphous valganciclovir hydrochloride under moisture controlled conditions with a median particle size below 250 micron.

### Example 2

### Preparation of Amorphous Valgancyclovir Hydrochloride

50 g valgancyclovir hydrochloride was dissolved in methanol (850 ml) and stirred to get a clear solution. The solution was passed through a micron filter to get a particle clear solution. The clear solution containing valgancyclovir hydrochloride was evaporated using a rotavaporator at a feed rate of about 5 to 15 ml/min at 10 to 30° C. at about 0 to 50 mbar vacuum, thus isolating a wet powdered form of amorphous valganciclovir hydrochloride under moisture controlled conditions. The wet mass was dried in vacuum oven to afford amorphous Valgancyclovir hydrochloride with a median particle size below 250 micron.

### Example 3

### Preparation of Amorphous Valgancyclovir Hydrochloride

50 g Valgancyclovir hydrochloride was dissolved in methanol (850 ml) and stirred to get a clear solution. The solution was passed through a micron filter to get a particle clear solution. The clear solution containing valgancyclovir hydrochloride was evaporated using a rotavaporator at 25 to 50° C. at about 0 to 200 mbar vacuum, thus isolating a wet powdered form of amorphous valganciclovir hydrochloride. The wet mass was dried in a vacuum oven to afford amorphous valgancyclovir hydrochloride with a median particle size below 250 micron.

### Example 4

### Preparation of Amorphous Valgancyclovir Hydrochloride

50 g valgancyclovir hydrochloride was dissolved in methanol (850 ml) and stirred to get a clear solution. The solution was passed through a micron filter to get a particle clear solution. The clear solution containing valgancyclovir hydrochloride was evaporated using a round bottom flask at 20 to 60° C. at about 0 to 50 mbar vacuum, thus isolating a wet powdered form of amorphous valgancyclovir hydrochloride. The wet mass was dried in a vacuum oven to afford amorphous valgancyclovir hydrochloride with a median particle size below 250 micron.

### Example 5

### Preparation of Amorphous Valgancyclovir Hydrochloride

50 g valgancyclovir hydrochloride was dissolved in methanol (850 ml) and stirred to get a clear solution. The solution was passed through a micron filter to get a particle clear solution. The clear solution containingvalgancyclovir hydrochloride was evaporated using a round bottom flask at 20 to 60° C. at about 0 to 150 mbar vacuum, thus isolating a wet powdered form of amorphous valgancyclovir hydrochloride. The wet mass was dried in a vacuum oven to afford amorphous valgancyclovir hydrochloride, which was further subjected to particle size reduction in order to get a median particle size below 100 micron.

### Example 6

### Process for Preparation of Amorphous Valgancyclovir Hydrochloride

500 g Valgancyclovir hydrochloride was dissolved in methanol (8500 ml) and the reaction mixture stirred to get a clear solution. The solution was passed through a micron filter to get a particle clear solution. The clear solution containing valgancyclovir hydrochloride was evaporated using an agitated thin film evaporator at a feed rate of about 5 to 15 ml/min at 50-70° C. at feed line and between 0-150 mbar vacuum. The system was cooled below 40° C. and unloaded under nitrogen controlled humidity, RH less than 35%, thus isolating a wet powdered form of amorphous valgancyclovir hydrochloride having methanol content less than 3% and moisture content less than 2% under moisture controlled conditions. The isolated wet mass was dried at 50-90° C. in a vacuum oven between 0-50 mbar. The system was cooled to below 40° C. and unloaded under nitrogen, controlled humidity, RH less than 35%, thus isolating a further wet powdered form of amorphous Valgancyclovir hydrochloride having a methanol content less than 3000 ppm and a moisture content and LOD less than 3% under nitrogen moisture controlled conditions RH less than 35% to afford amorphous valgancyclovir hydrochloride (400 g) under moisture controlled conditions with a particle size below 100 micron. The product was then packed in the condition mentioned below:

Packing mode: The material is packed in a HMLDPE bag under vacuumised nitrogen sealing at 30±5% RH. It is then inserted in triple laminated aluminum bag under vacuumised nitrogen sealing with two silica gel sachets and one molecular sieve sachet. It is then inserted into a triple laminated aluminum bag under vacuumised nitrogen sealing with two silica gel sachets and one molecular sieve sachet. Such bags are further packed in HDPE containers.

HPLC Purity Related substances NMT 1.5%; Enantiomeric purity NLT: 98.5%; Diastereomeric ratio: between (45:55) to (55:45)

### Example 7

### Process for Particle Size Reduction of Amorphous Valgancyclovir Hydrochloride

At RH below 35% semidried/dried amorphous valgancyclovir hydrochloride is passed through a screen to separate coarse and fine particles below 200 micron and coarse particles are reduced by standard attrition mills to 200 microns.

We claim:

1. Amorphous valgancyclovir hydrochloride having a median particle size less than 100 μm.

2. A process for the preparation of an amorphous valgancyclovir hydrochloride having a median particle size of less than 100 μm comprising the steps of:
a) dissolving valgancyclovir hydrochloride in at least one solvent,
b) removing the at least one solvent, and
c) drying the wet mass to isolate the amorphous valgancyclovir hydrochloride having a median particle size below 100 μm.

3. The process according to claim 2, wherein the at least one solvent is selected from C₁-C₆ alcohols, ketones, hydrocarbons, water and mixtures thereof.

4. The process according to claim 3 wherein the C₁-C₆ alcohols are methanol, ethanol, n-propanol, isopropanol, n-butanol, isobutanol, and t-butanol and the ketones are acetone, propanone and 2-butanone.

5. The process according to claim 2, wherein the at least one solvent is removed by agitated thin film evaporation or vacuum distillation.

6. The process according to claim 5, wherein when solvent removal is by evaporation, the evaporation is carried out at a feed rate of about 1 to 50 ml/min and a temperature of about 10 to 100° C. using 0 to 200 mbar vacuum.

7. The process according to claim 2, wherein wet mass is dried by tray drying, spray drying, vacuum drying or freeze-drying.

8. Amorphous valgancyclovir hydrochloride having a HPLC purity greater than 98.5%.

9. Amorphous Valgancyclovir hydrochloride having at least one of the characteristics selected from the group consisting of
i) an enantiomeric purity more than 98.5%;
ii) a diastereomeric ratio of about (45:55) to (55:45);
iii) A loss on drying of less than 3%;
iv) A moisture content of less than 3%;
v) A residual solvent content less than 3000 ppm; and
vi) a median particle size less than 100 μm.

10. A process for the preparation of an amorphous valgancyclovir hydrochloride having a median particle size of less than 100 μm comprising the steps of:
a) dissolving valgancyclovir hydrochloride in at least one solvent,
b) removing the at least one solvent,
c) drying the wet mass to isolate an amorphous valgancyclovir hydrochloride having a median particle size of below 250 μm, and
d) reducing the particle size to a median particle size below 100 μm.

11. A method for packing an amorphous valgancyclovir hydrochloride, comprising the steps of:
a) placing amorphous valgancyclovir hydrochloride in a HMLDPE bag under an inert atmosphere at 30±5% RH,
b) placing the HMLDPE bag in a triple laminated aluminum bag under an inert atmosphere with two silica gel sachets and one molecular sieve sachet,
c) placing the contents of step b in a triple laminated aluminum bag under an inert atmosphere with two silica gel sachets and one molecular sieve sachet; and
d) packing the contents of step c in a closed high density polyethylene (HDPE) container.
